(19) United States
(12) Patent Application Publication
(10) Pub. No.: US 2026/0325033 A1
(43) Pub. Date: Oct. 22, 2026

(54) SYSTEM AND METHOD FOR FILTERING INTERFERENCE GENERATED BY AN ELECTROSURGICAL UNIT FROM AN ELECTROCARDIOGRAM SIGNAL

(71) Applicants: Draeger Medical Systems, Inc., Andover, MA (US); ERASMUS MEDICAL CENTER, GD Rotterdam (NL)

(72) Inventors: Clifford Risher-Kelly, Wells, ME (US); Ruwen Zhang, Andover, MA (US); Ryan Forde, Pelham, NH (US); Martijn Jansen, Rotterdam (NL); René Rudolf Peter Maria Hagenouw, Utrecht (NL)

(21) Appl. No.: 19/106,934

(22) PCT Filed: Aug. 26, 2022
(86) PCT No.: PCT/IB2022/058022

§ 371 Date: Feb. 26, 2025

(51) Int. Cl. A61B 5/308; A61B 5/28; A61B 5/339

(52) U.S. Cl. CPC A61B 5/308; A61B 5/28; A61B 5/339

(57) ABSTRACT

A system and method for filtering interference generated by an interference signal, such as electrosurgical instrument (ESU), from a biopotential signal, such as an electrocardiogram (ECG) signal displayed on a patient monitor. The system includes acquisition circuitry to acquire an incoming electrical signal from a patient that includes a biopotential signal and interference signal. The system includes attenuation circuitry to attenuate the voltage to a level below a predetermined clipping threshold of front-end protective circuitry. A filter is provided to remove the interference generated by the interference signal from the attenuated incoming electrical signal to obtain a filtered biopotential signal having reduced interference. Gain circuitry is provided to amplify the filtered biopotential signal to obtain an output biopotential signal suitable for displaying on the patient display monitor.

### CROSS REFERENCCE TO RELATED APPLICATION

This application is a U.S. National Stage Patent Application based on PCT No. PCT/IB/2022/058022, filed Aug. 26, 2022, and claiming priority, the entire contents of which are hereby incorporated by reference as though fully set forth verbatim herein.

### FIELD OF THE INVENTION

The present disclosure relates generally to methods and systems for removing unwanted noise or interference from an electrocardiogram signal.

### BACKGROUND

Patients in intensive care units (ICUs) or critical care units (CCUs) may require continuous monitoring of electrocardiogram (ECG) signals, as well as other biopotential signals, such as electroencephalography (“EEG”) signals and electromyography (“EMG”) signals. ECG waveforms on patient monitors can be viewed and interpreted by qualified medical staff or automatically processed by algorithms in the patient monitors to identify, or detect, specific features or characteristics of the waveform.

Patient monitors are used to provide feedback to clinicians regarding real-time patient health. The patient monitors are configured to display output relating to real-time physiological parameters or vital signs of the patient. Clinical professionals monitor the display output to determine the current status of the patient's health and to diagnose current patient conditions. In addition to visual display, the patient monitors may also be configured to generate audible output (e.g., alarms) if a particular physiological parameter or vital sign being monitored falls outside a threshold range to alert the clinical professionals of an unsafe condition that may require medical assistance or attention.

One example of a biopotential signal that is commonly monitored and displayed on patient monitors is an ECG waveform that is indicative of the electrical activity in the heart. The ECG waveform can be monitored by clinical professionals to determine whether any deviations or abnormalities occur that may be indicative of an unsafe and potentially life-threatening condition (e.g., atrial fibrillation, ventricular tachycardia, heart disease, cardiac arrest) that may require immediate medical attention or therapeutic treatment. The ECG waveform can be used to evaluate heart rate, rhythm, and other cardiac abnormalities and to make diagnoses. Accordingly, it is desirable that the ECG waveform that is displayed on the patient monitor is clean and uncorrupted so as not to generate false alarms or prompt medical treatment that is not warranted and may cause harm to the patient.

It is not uncommon for other electrical signals to be picked up by biopotential sensors during detection of a biopotential signal. Accordingly, it is known to provide filtering for the purpose of making the biopotential signal easier to read and interpret. Filtering noise from a biopotential signal is particularly challenging if the interfering signal has a significantly greater amplitude than that of the biopotential signal because the interfering signal will be clipped by the front end protective circuitry present in most devices used to gather biopotential signals, such as patient monitors. Once the interfering signal is clipped, it is extremely difficult to filter.

For example, electrosurgical units (ESUs) are routinely used in operating rooms and are known to interfere with the monitoring of a patient's biopotential signals. Unfortunately, the presence of electromagnetic energy generated by operation of an ESU can cause unwanted interference with biopotential signals. The terms ESU signal, ESU interference, and ESU noise are used interchangeably herein and are all intended to refer to noise in a biopotential signal resulting from an ESU being used on the patient. For example, noise from the use of an ESU on the patient can cause an ECG waveform to be difficult or impossible to read, decipher or interpret. Accordingly, attempts have been made to filter ECG signals in an attempt to reduce noise resulting from use of an ESU.

Filtering an ECG signal to remove interference from an ESU can be challenging due to the difference between the amplitude of an ECG signal (typically no more than 1 mV) and the amplitude of the signal applied to a patient's body when an ESU is in use (often between 10 and 100V). A typical patient monitoring system ECG sensor interface (or “front end”) is designed to receive an ECG signal, and therefore, often has a maximum signal amplitude of about ±2V. Accordingly, when an ESU signal is applied to the patient, the signal received by the ECG interface can exceed the capacity of the ECG sensor interface, which will cause the signal to be clipped by front end protection circuit, meaning that portions of the signal that are above the maximum amplitude of the ECG sensor interface are removed from the signal. It is very difficult to filter the clipped signal using known filtering techniques because the clipped signal is nonlinear.

The present invention utilizes a novel technique to filter interference originating from the ESU to render a clean and uncorrupted ECG waveform to enable a surgeon to continue using the ESU in the operating room while monitoring the patient's ECG.

### SUMMARY

In embodiments described herein, an input biopotential signal, which includes an interference signal, is attenuated to a level sufficient to avoid clipping of the input biopotential signal by the front-end protection circuit. The signal is then filtered and amplified to its original level before being displayed on the graphical user interface of a patient monitor.

The following are additional aspects of the invention.

Aspect 1: A method of operating a patient monitor, the method comprising:
(a) receiving an input biopotential signal from at least one biopotential electrode that is electrically coupled to a patient, the input bipotential signal having an input voltage;
(b) attenuating the input biopotential signal to produce an attenuated biopotential signal having an attenuated voltage, the attenuated voltage being less than the input voltage;
(c) filtering the attenuated biopotential signal to reduce any interference generated by an electrosurgery unit (ESU), thereby producing a filtered biopotential signal;
(d) amplifying the filtered biopotential signal to obtain an output biopotential signal having an amplified voltage; and
(e) displaying the output biopotential signal.

Aspect 2: The method of aspect 1, wherein step (b) comprises attenuating the input biopotential signal by a predetermined voltage attenuation ratio.

Aspect 3: The method of aspect 2, wherein the predetermined voltage attenuation ratio is at least 20.

Aspect 4: The method of any one of aspects 2 to 3, wherein step (d) comprises amplifying the filtered biopotential signal by a voltage amplification ratio that is equal to the voltage attenuation ratio.

Aspect 5: The method of any one of aspects 1 to 4, wherein the input biopotential signal is an electrocardiogram (ECG).

Aspect 6: The method of any one of aspects 1 to 5, further comprising:
(f) detecting the presence of an ESU signal.

Aspect 7: The method of aspect 6, wherein step (c) is only performed if step (f) is performed and an ESU signal is detected.

Aspect 8: The method of aspect 6, wherein steps (b) through (d) are only performed if step (f) is performed and an ESU signal is detected.

Aspect 9: The method of any one of aspects 6 to 8, wherein step (f) is performed in response to at least one selected from the group of (a) a user selectable switch is activated and (b) a location device determines that the patient monitor is located in an operating room.

Aspect 10: The method of any one of aspects 6 to 8, wherein step (f) is performed in response to the location device determining that the patient monitor is docked to a monitor mount located in an operating room.

Aspect 11: The method of any one of aspects 1 to 10, further comprising performing step (b) before feeding the attenuated biopotential signal into a front end protective circuit of the physiological monitoring device, the front end protective circuit having a clipping voltage.

Aspect 12: The method of aspect 11, wherein the input voltage exceeds the clipping voltage when the ESU is being used on the patient.

Aspect 13: The method of any one of aspects 1 to 12, further comprising:
(g) processing the output biopotential signal before performing step (e).

Aspect 14: The method of any one of aspects 1 to 12, wherein the input biopotential signal further comprises noise generated by the use of the ESU on the patient.

Aspect 15: The method of any one of aspects 1 to 12, further comprising:
(h) measuring an impedance of each of the at least one biopotential electrode;
(i) adjusting an attenuator circuit based on the impedance measured in step (h) to maintain the predetermined voltage attenuation ratio.

Aspect 16: The method of aspect 15, further comprising:
(j) repeating steps (h) and (i) at a predetermined time interval.

Aspect 17: The method of any one of aspects 15 to 16, further comprising performing the impedance measurement of step (h) using AC impedance measurement when an ESU signal is detected.

Aspect 18: A system comprising:
a sensor interface adapted to receive an input biopotential signal from at least one biopotential electrode that is electrically coupled to a patient, the input biopotential signal having an input voltage;
an attenuator electrically coupled to an output of the sensor interface, the attenuator being adapted to receive the input biopotential signal and to produce an attenuated biopotential signal having an attenuated voltage that is reduced from the input voltage by a voltage attenuation ratio;
an analog to digital convertor electrically coupled to an output of the attenuator, the analog to digital converter being adapted to convert the attenuated biopotential signal to a digital attenuated biopotential signal;
a protection circuit electrically coupled to an output of the analog to digital convertor, the protection circuit adapted to remove any portions of the digital attenuated biopotential signal that exceed a clipping voltage and to produce a clipped biopotential signal;
at least one filter electrically coupled an output of the protection circuit, the at least one filter adapted to reduce any ESU signal noise in the clipped biopotential signal and to produce a filtered biopotential signal, the ESU signal noise being generated by use of an electrosurgical instrument (ESU) on the patient when the input biopotential signal is being received;
an amplifier electrically coupled to an output of the at least one filter, the amplifier being adapted to receive the filtered biopotential signal and to produce an amplified biopotential signal having an amplified voltage that is increased from the attenuated voltage by a voltage amplification ratio; and
a display electrically coupled to the amplifier, the display being adapted to provide a visual representation of the amplified biopotential signal or a biopotential signal that is derived from the amplified biopotential signal.

Aspect 19: The system of aspect 18, wherein the attenuator is adapted to attenuate the input biopotential signal by a voltage attenuation ratio of at least 20.

Aspect 20: The system of aspect 19, wherein the amplifier is adapted to amplify the filtered biopotential signal by a voltage amplification ratio that is equal to the voltage attenuation ratio.

Aspect 21: The system of any one of aspects 18 to 19, wherein the sensor interface comprises an electrocardiogram (ECG) sensor interface.

Aspect 22: The system of aspect 21, wherein the at least one biopotential electrode comprises a plurality of ECG electrodes.

Aspect 23: The system of any one of aspects 18 to 22, further comprising an ESU detection unit having an active state and an inactive state, the ESU detection unit being adapted to detect the presence of an ESU signal in the input biopotential signal only in the active state.

Aspect 24: The system of aspect 23, wherein the ESU detection unit is further adapted to enable the input biopotential signal to bypass the attenuator, the at least one filter, and the amplifier when the ESU detection unit is in the active state and an ESU signal is detected.

Aspect 25: The system of aspect 24, further comprising a location detection unit that is adapted to place the ESU detection unit in an active state in response to at least one selected from the group of (a) a user selectable switch is activated and (b) the location detection unit determines that the patient monitor is located in an operating room.

Aspect 26: The system of any one of aspects 18 to 25, wherein the attenuator includes a variable resistor.

Aspect 27: The system of any one of aspects 18 to 26, further comprising an AC impedance detection circuit adapted to measure the impedance of each of the at least one biopotential electrode.

Aspect 28: A method comprising:
(a) receiving an input biopotential signal from at least one biopotential electrode that is electrically coupled to a patient and a physiological monitoring device, the input bipotential signal having an input voltage;
(b) detecting the presence of an interference signal in the input biopotential signal;
(c) attenuating the input biopotential signal by a predetermined voltage attenuation ratio to produce an attenuated biopotential signal having an attenuated voltage, the attenuated voltage being less than the input voltage and less than a clipping threshold of a protective circuit of the physiological monitoring device;
(d) filtering the attenuated biopotential signal to remove at least one component of the interference signal, thereby producing a filtered biopotential signal;
(e) amplifying the filtered biopotential signal to obtain an output biopotential signal having an amplified voltage; and
(f) displaying the output biopotential signal.

Aspect 29: The method of aspect 28, further comprising:
(g) performing steps (c) through (e) only when an interference signal is detected in step (b).

Aspect 30: The method of aspect 29, wherein step (b) is performed in response to at least one selected from the group of: a user selectable switch is activated and a location device determines that the patient monitor is located in an operating room.

Aspect 31: The method of aspect 29, wherein step (b) is performed in response to the location device determines that the patient monitor is docked to a monitor mount located in an operating room.

Aspect 32: The method of any one of aspects 28 to 31, further comprising:
(h) measuring an electrode impedance for each of the at least one biopotential electrodes; and
(i) adjusting an attenuator resistance based on the measured electrode impedance in order to achieve the predetermined voltage attenuation ratio.

Aspect 33: The method of aspect 32, further comprising:
(j) repeating steps (h) and (i) at a predetermined time interval.

Aspect 34: The method of any one of aspects 32 to 33, further comprising performing the impedance measurement of step (h) using AC impedance measurement when an ESU signal is detected.

Aspect 35: The method of any one of aspects 28 to 34, step (c) further comprises attenuating the input biopotential signal by a predetermined voltage attenuation ratio to produce an attenuated biopotential signal having an attenuated voltage, the attenuated voltage being less than the input voltage and less than a clipping threshold of a protective circuit of the physiological monitoring device.

Aspect 36: The method of any one of aspects 28 to 34, wherein the at least one biopotential electrode is an ECG electrode.

Aspect 37: The method of any one of aspects 28 to 34, wherein step (c) comprises attenuating the input biopotential signal by a predetermined voltage attenuation ratio of at least 20.

Aspect 38: The method of aspect 37, wherein step (e) comprises amplifying the filtered biopotential signal by a voltage amplification ratio that is equal to the voltage attenuation ratio.

### BRIEF DESCRIPTION OF THE DRAWINGS

The invention will be described in conjunction with the following drawings in which like reference numerals designate like elements and wherein:

FIG. 1 is a schematic diagram showing an exemplary physiological monitoring system illustrating the major components of the signal processing system of the present disclosure;

FIG. 2 is a diagram illustrating the signal processing components of the signal processing system of the present disclosure;

FIG. 3 is amplitude (mV) vs. time (seconds) graph showing an exemplary signal generated by an electrocardiogram;

FIG. 4 is an amplitude (V) vs. time (seconds) graph showing an exemplary signal comprising an ECG signal component and an interfering ESU signal component;

FIG. 5 is an amplitude (V) vs. time (microseconds) graphs showing an exemplary ESU signal after being clipped at ±2V;

FIG. 6 is an amplitude spectrum (relative) vs. frequency (Hz) graph showing frequency components generated by clamping an ESU signal;

FIG. 7 is an amplitude (mV) vs. time (seconds) graph of the signal shown in FIG. 4 after being filtered;

FIG. 8 is a schematic diagram showing an exemplary attenuator circuit with a variable resistor; and

FIG. 9 is a flow diagram showing an exemplary method of method of operating a physiological monitoring system to filter an ESU signal from an ECG signal.

### DETAILED DESCRIPTION

The following disclosure is presented to provide an illustration of the general principles of the present invention and is not meant to limit, in any way, the inventive concepts contained herein. Moreover, the particular features described in this section can be used in combination with the other described features in each of the multitude of possible permutations and combinations contained herein.

All terms defined herein should be afforded their broadest possible interpretation, including any implied meanings as dictated by a reading of the specification as well as any words that a person having skill in the art and/or a dictionary, treatise, or similar authority would assign particular meaning. Further, it should be noted that, as recited in the specification and in the claims appended hereto, the singular forms “a,” “an,” and “the” include the plural referents unless otherwise stated. Additionally, the terms “comprises” and “comprising” when used herein specify that certain features are present in that embodiment, but should not be interpreted to preclude the presence or addition of additional features, components, operations, and/or groups thereof.

As used herein, the term “interference signal” means an electrical signal detected by a biopotential sensor connected to a patient (a) that is not a biopotential signal, (b) has a frequency of 40 Hz or greater and (c) an amplitude that exceeds the dynamic range of the instrument to which the biopotential sensor is connected. Examples of interference signals include ESU noise, powerline interference (PLI) noise, white gaussian noise (often generated by wireless transmission of ECG signals), and EMG noise.

As used herein, the term “biopotential signal” means electrical signals (voltages) that are generated, either directly or indirectly, by physiological processes occurring within the human body. Examples of biopotential signals include, but are not limited to, electroencephalography (“EEG”) signals, electromyography (“EMG”) signals, electrocardiogram (ECG) signals, neuromuscular transmission (NMT) signals, and pulse oximetry (SpO2) signals.

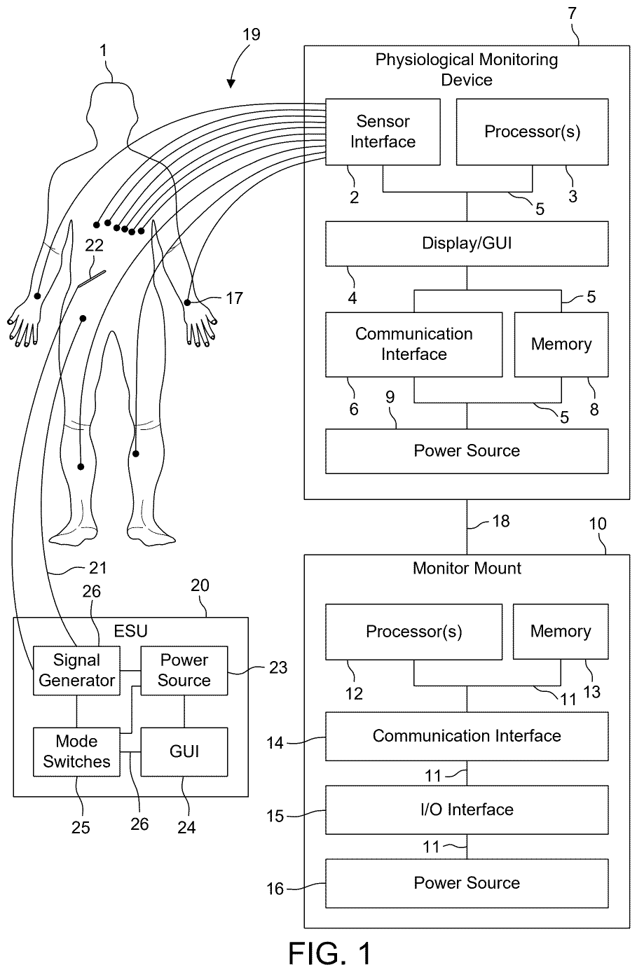

FIG. 1 is a schematic diagram of an exemplary physiological monitoring system 19 (also referred to as a patient monitor) with a plurality of sensors 17 connected to a patient 1 and an exemplary ESU 20. For example, the plurality of sensors could include an ECG sensor having a plurality of surface ECG leads (electrodes) for detecting and analyzing ECG waveforms. As illustrated, the system includes a physiological monitoring device 7 capable of receiving physiological data from the sensors 17, and a monitor mount 10 to which the physiological monitoring device 7 can be mounted or docked.

The physiological monitoring device 7 is, for example, a patient monitor implemented to monitor various physiological parameters of the patient 1 via the sensors 17. The physiological monitoring device 7 includes a sensor interface 2, one or more processors 3, a display/GUI 4, a communications interface 6, a memory 8, and a power source 9. The sensor interface 2 can be implemented in software or hardware and used to connect via wired and/or wireless connections to one or more physiological sensors and/or medical devices 17 for gathering physiological data from the patient 1. The data signals from the sensors 17 include, for example, data related to an electrocardiogram (ECG), non-invasive peripheral oxygen saturation (SpO2), non-invasive blood pressure (NIBP), temperature, and/or end tidal carbon dioxide (etCO2), apnea detection, neuromuscular transmission (“NMT”), electroencephalogram (“EEG”), and brain monitoring external (“BISx”), and other similar physiological data.

The communications interface 6 allows the physiological monitoring device 7 to directly or indirectly (via, for example, the monitor mount 10) communicate with one or more computing networks and devices (not shown). The communications interface 6 can include various network cards, interfaces or circuitry to enable wired and wireless communications with such computing networks and devices. The communications interface 6 can also be used to implement, for example, a Bluetooth connection, a cellular network connection, and/or a WiFi connection. Other wireless communication connections implemented using the communications interface 6 include wireless connections that operate in accordance with, but are not limited to, IEEE802.11 protocol, a Radio Frequency for Consumer Electronics (RF4CE) protocol, ZigBee protocol, and/or IEEE802.15.4 protocol.

Additionally, the communications interface 6 can enable direct (i.e., device-to-device) communications (e.g., messaging, signal exchange, etc.) such as from the monitor mount 10 to the physiological monitoring device 7 using, for example, a USB connection. The communications interface 6 can also enable direct device-to-device connection to other devices such as to a tablet, PC, or similar electronic device, or to an external storage device or memory.

The power source 9 can include a self-contained power source such as a battery pack and/or include an interface to be powered through an electrical outlet (either directly or by way of the monitor mount 10). The power source 9 can also be a rechargeable battery that can be detached allowing for replacement. In the case of a rechargeable battery, a small built-in back-up battery (or super capacitor) can be provided for continuous power to be provided to the physiological monitoring device 7 during battery replacement. Communication between the components of the physiological monitoring device 7 (e.g., 2, 3, 4, 6, 8, and 9) are established using an internal bus 5.

As shown in FIG. 1, the physiological monitoring device 7 is connected to the monitor mount 10 via a connection 18 that establishes a communication connection between, for example, the respective communications interfaces 6, 14 of the devices 7, 10. The connection 18 enables the monitor mount 10 to detachably secure the physiological monitoring device 7 to the monitor mount 10. In this regard, “detachably secure” means that the monitor mount 10 can secure the physiological monitoring device 7, but the physiological monitoring device 7 can be removed or undocked from the monitor mount 10 by a user when desired. The connection 18 may include, but is not limited to, a universal serial bus (USB) connection, parallel connection, a serial connection, coaxial connection, a High-Definition Multimedia Interface (HDMI) connection, or other similar connection known in the art connecting to electronic devices.

The monitor mount 10 includes one or more processors 12, a memory 13, a communications interface 14, an I/O interface 15, and a power source 16. The one or more processors 12 are used for controlling the general operations of the monitor mount 10. The memory 13 can be used to store any type of instructions associated with algorithms, processes, or operations for controlling the general functions and operations of the monitor mount 10.

The communications interface 14 allows the monitor mount 10 to communicate with one or more computing networks and devices (e.g., the physiological monitoring device 7). The communications interface 14 can include various network cards, interfaces or circuitry to enable wired and wireless communications with such computing networks and devices. The communications interface 14 can also be used to implement, for example, a Bluetooth connection, a cellular network connection, and a WiFi connection. Other wireless communication connections implemented using the communications interface 14 include wireless connections that operate in accordance with, but are not limited to, IEEE 802.11 protocol, a Radio Frequency For Consumer Electronics (RF4CE) protocol, ZigBee protocol, and/or IEEE 802.15.4 protocol.

The communications interface 14 can also enable direct (i.e., device-to-device) communications (e.g., messaging, signal exchange, etc.) such as from the monitor mount 10 to the physiological monitoring device 7 using, for example, a USB connection, coaxial connection, or other similar electrical connection. The communications interface 14 can enable direct connections (i.e., device-to-device) to other devices such as to a tablet, PC, or similar electronic device, or to an external storage device or memory.

The I/O interface 15 can be an interface for enabling the transfer of information between monitor mount 10, one or more physiological monitoring devices 7, and external devices such as peripherals connected to the monitor mount 10 that need special communication links for interfacing with the one or more processors 12. The I/O interface 15 can be implemented to accommodate various connections to the monitor mount 10 that include, but is not limited to, a universal serial bus (USB) connection, parallel connection, a serial connection, coaxial connection, a High-Definition Multimedia Interface (HDMI) connection, or other known connection in the art connecting to external devices.

The power source 16 can include a self-contained power source such as a battery pack and/or include an interface to be powered through an electrical outlet. The power source 16 can also be a rechargeable battery that can be detached allowing for replacement. Communication between the components of the monitor mount 10 (e.g., 12, 13, 14, 15 and 16) are established using an internal bus 11.

The ESU 20 comprises a signal generator 26 that supplies an electrical signal to a handpiece 22, which is used to perform varies surgical functions to the tissue of the patient 1, such as cutting, coagulating, ablating, and shrinking, depending upon the nature of the electrical signal supplied by the signal generator 26. The handpiece 22 is the active electrode for applying the electrical signal. In this embodiment, a return electrode 21 is applied to the surface of the patient's skin in close proximity to the site where the handpiece 22 is being used. The surgical function applied by the handpiece 22 can be changed by changing the voltage and/or frequency of the signal. This is accomplished using one or more mode switches 25. The ESU 20 also preferably includes a graphical user interface 24 which displays various information concerning the state of operation of the ESU 20, such as which surgical function is active. A power source 23 provides power to the other components of the ESU. The power source is preferably an interface powered through an electrical outlet. Communication between components of the ESU is established using an internal bus 26.

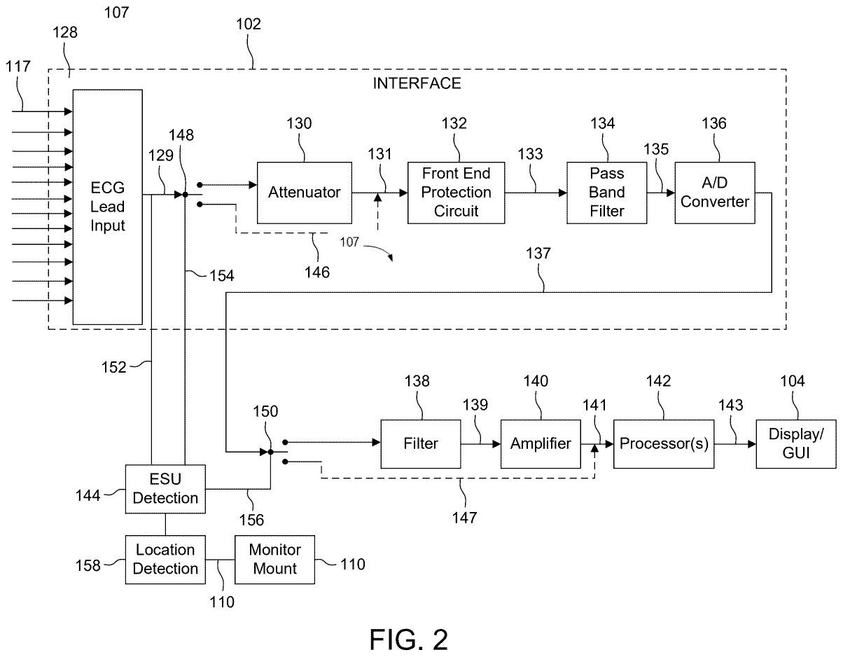

FIG. 2 is an exemplary embodiment of portions of a physiological monitoring device 107 that are adapted to receive, process, and display an ECG signal from a patient and reduce interference generated by an ESU signal. It should be noted that many of the functional elements that are schematically shown in FIG. 2 may be executed with hardware and/or software. In addition, there may be additional functional elements present in the physiological monitoring device 107. The embodiment is intended to be exemplary could be applied advantageously to the filtering of other interference signals from other type of biopotential signals.

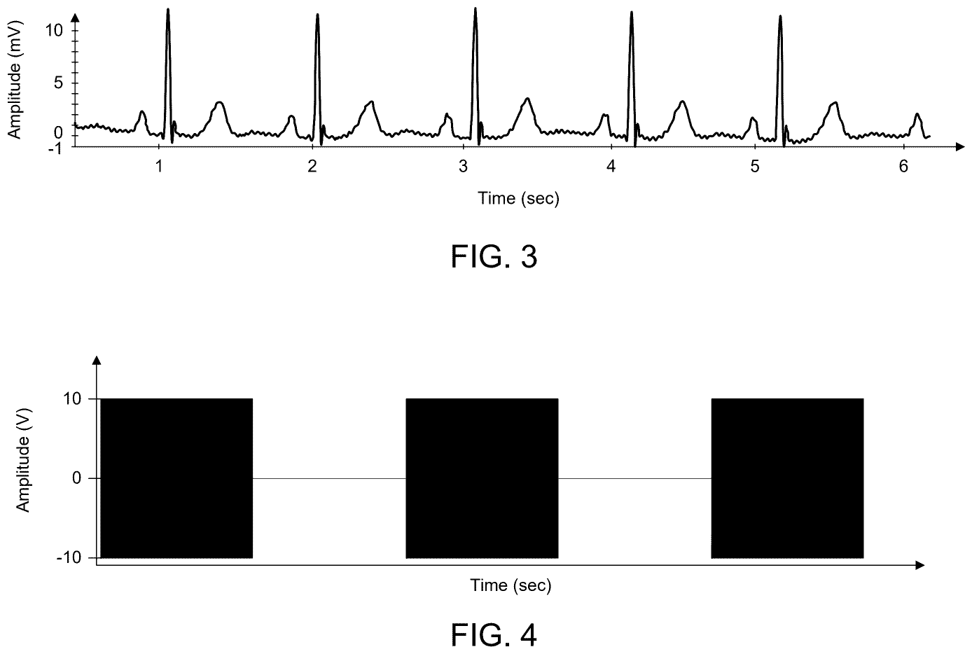

In this embodiment, the sensor interface 102 comprises a 12-lead ECG interface 128. The ECG leads 117 are represented by the arrows pointing into the interface 128. The input biopotential signal 129 (in this embodiment, an ECG signal) is received from the ECG interface 128. FIG. 3 shows an example of a “clean” ECG input biopotential signal, i.e., without any significant interference from an ESU or other sources. FIG. 4 shows an input biopotential signal having both an ECG signal and ESU noise/interference. As can be seen in FIG. 4, the relatively high amplitude and frequency of the ESU signal, “drown out” the ECG and render the signal unusable for purposes of viewing the ECG component of the signal.

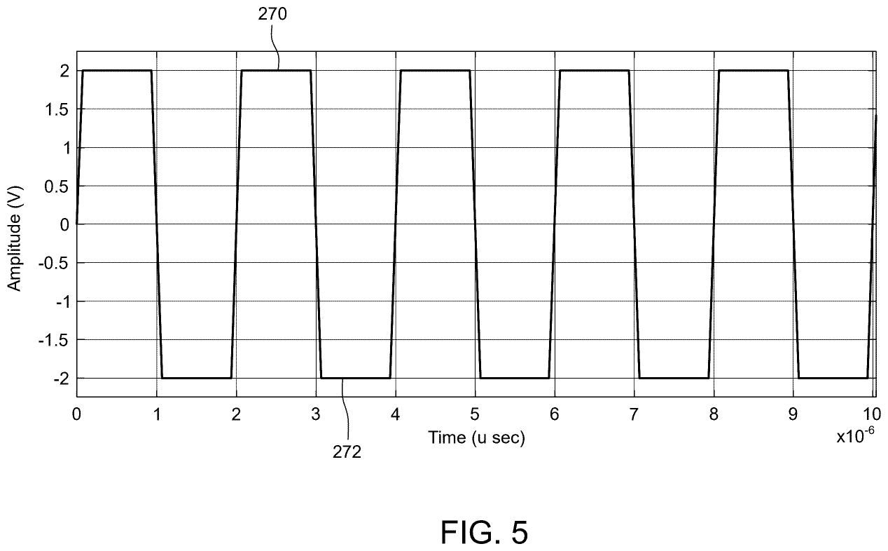

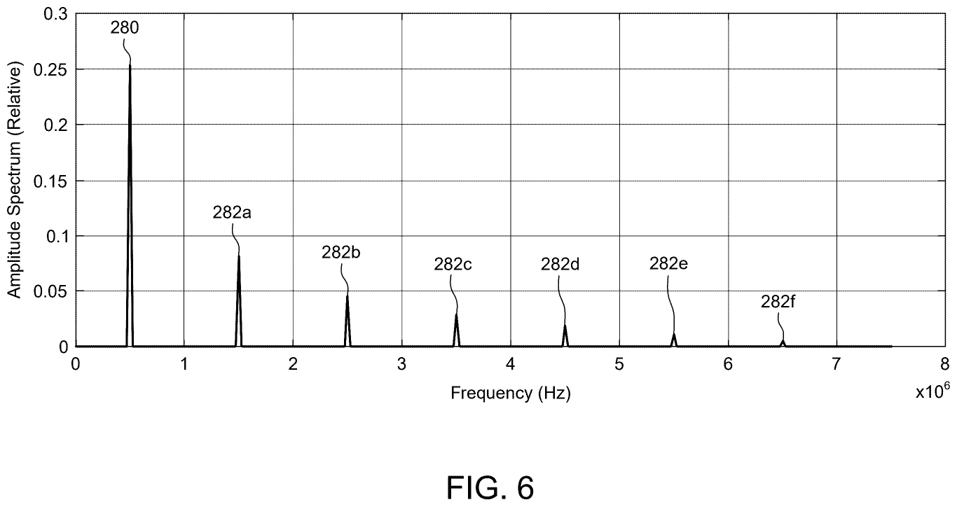

As noted above, an ESU is most commonly employed during surgery in an operating room. During use, the ESU will generate significant electromagnetic interference, which will be detected by the ECG leads 117, and therefore, will become part of the input biopotential signal 129. As is common in the industry, the physiological monitoring device 107 includes protection circuitry (front end protection circuit 132) which is intended to protect other circuitry against damage from a high voltage signal or surge. Accordingly, the input biopotential signal 129 will have a voltage considerably greater than that of the protection circuitry and the signal will be clipped by the protection circuitry (compare FIG. 3 to FIG. 4). FIG. 5 shows the ESU component of the biopotential signal of FIG. 4 after being clipped by a front end protection circuit 132. As can be seen in FIG. 5, the peaks 270 and valleys 272 of the ESU signal (which is typically a sine wave) are flattened (clipped) at ±2V. In addition, clipping of the biopotential signal of FIG. 4 will often generate additional high frequency components. An example of this is shown in FIG. 6. In FIG. 6, clamping of a 500 Hz ESU signal 280 generates several high frequency components 282a through 282f, which range from 1500 Hz to 6500 Hz. Currently, there are no techniques available to effectively recover a clipped signal, then filter the ECG signal from ESU interference.

In order to avoid such clipping, the input biopotential signal 129 is passed to an attenuator 130, which attenuates the input biopotential signal 129 to produce an attenuated biopotential signal 131. In this embodiment, the attenuator 130 preferably reduces the voltage amplitude of the input biopotential signal 129 by a voltage attenuation ratio of 20. A voltage attenuation ratio of 20 is a compromise in that some clipping of the input biopotential signal 129 will still occur, but a higher voltage attenuation ratio would result in an unacceptably low signal to noise ratio in the output signal. In other embodiments, attenuator 130 preferably reduces the voltage amplitude of the input biopotential signal 129 by a voltage attenuation ratio of at least 20, more preferably, by at least 50, and most preferably, by at least 80. The attenuator 130 could comprise any suitable attenuator circuit, such as a resistor divider.

The attenuated biopotential signal 131 is then passed through a front end protective circuit 132, which removes (“clips”) any portion of the attenuated biopotential signal 131 having a greater voltage than the maximum voltage (positive or negative) of the circuit 132 (also referred to as a “voltage limit”), thereby producing a clipped biopotential signal 133. It is common for the voltage limit of a front end protective circuit 132 for a physiological monitoring device 107 to be between ±2.0V and 2.7V. As noted above, the purpose of attenuating the input biopotential signal 129 prior to passing through the front end protective circuit 132 is to reduce clipping of ESU signal noise in the input biopotential signal 129 by the front end protective circuit 132 because it is much more difficult to filter ESU signal noise from the biopotential signal once it has been clipped.

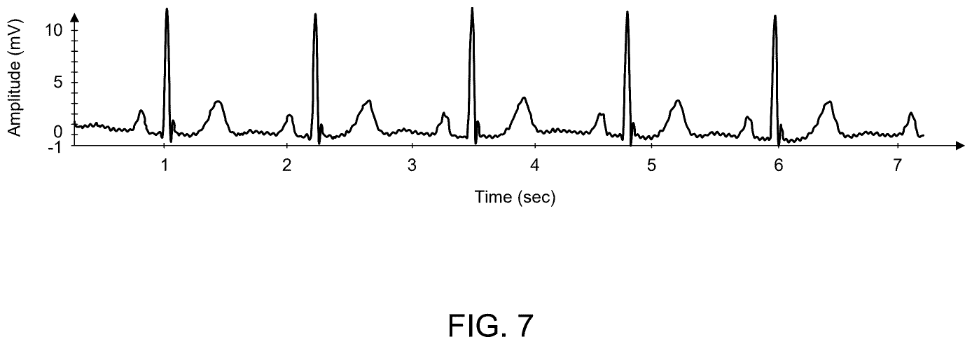

The clipped biopotential signal 133 is then converted to a digital biopotential signal 137 by an analog to digital convertor 136. Any suitable analog to digital convertor 136 could be used, such as a Texas Instruments ADS 1298 24-bit analog to digital convertor. The digital biopotential signal 137 is then filtered by filter(s) 138 to reduce ESU signal noise and produce a filtered biopotential signal 139. Filtration of the biopotential signal 139 may be performed using any suitable means, including hardware and/or software-based filtration. Examples of suitable filtration techniques can be found in U.S. Pat. No. 6,246,902, entitled Lead Set Filter for a Patient Monitor, which is incorporated herein by reference as if fully set forth. Preferably, the filtration includes a high pass filter that will remove any direct current components of the biopotential signal 139. An example of a filtered bipotential signal 139 is shown in FIG. 7.

The filtered bipotential signal 139 is then amplified, using amplifier 140, to produce an amplified biopotential signal 141. Preferably, the amplified biopotential signal 141 is substantially the same signal level as the input biopotential signal 129. In other words, it is preferrable that the voltage attenuation ratio applied to by the attenuator 130 be substantially the same as the voltage amplification ratio applied by the amplifier 140.

The amplified biopotential signal 141 is then further processed by one or more processors 142 and, optionally, other circuitry, then displayed on a graphical user interface 104. As is known in the art, the amplified biopotential signal 141 could be processed in multiple parallel signal paths for different purposes, such as an analog output, a QRS sync marker, to provide adaptive filtering, and an ECG waveform display.

It is also desirable that circuitry that provides ESU filtering functionality, i.e., the attenuator 130, the ESU signal filters 138, and the amplifier 140 (hereinafter “ESU filtering circuitry”), be active only when the ESU unit is being actively used on the patient. In this embodiment, this is accomplished by activating the ESU filtering circuitry only when an ESU signal is detected by an ESU detection unit 144 and only activating the ESU detection unit 144 when the patient is located in an operating room.

Examples of suitable circuitry for the ESU detection unit 144 are disclosed in U.S. Pat. No. 10,568,678, entitled Neutral Drive Feedback Loop Compensation for Detected Electrosurgical Unit Signal, and U.S. Pat. No. 11,103,190, entitled Circuits and Methods for Electrosurgical Unit Signal Detection, which are incorporated by reference as if fully set forth. An exemplary ESU detection unit 144 is shown schematically in FIG. 2. A lead 152 is electrically connected to the input biopotential signal 129 to detect ESU interference. If ESU interference is detected, then the ESU filtering circuitry is activated or engaged. The bypassing of the ESU filtering circuitry is shown schematically in FIG. 2 by bypass circuits 146 and 147. Switches 148 and 150, which are controlled by leads 154 and 156, respectively, send signals through the bypass circuits 146 and 147 when ESU interference is not detected or if the ESU detection circuitry 144 is inactive.

Preferably, the ESU detection unit 144 is only active when the patient is located in a room in which an ESU unit is likely to be used, such as an operating room. In this embodiment a location detection unit 158 is provided. The location detection unit 158 is electrically connected to the ESU detection unit 144 and places the ESU detection unit 144 in an active or inactive mode. There are many inputs that could be used, either alone or in combination, by the location detection unit 158 to determine that the ESU detection unit 144 should be active. The input could be as simple as a user-activated on-off switch in the graphical user interface 104. Other possible inputs include a sensor 162 that detects when the patient monitor is docked to a monitor mount 110 located in an operating room or a GPS-enabled sensor (not shown) capable of detecting when the patient monitor is in an operating room.

In some applications, it may be desirable to adjust the attenuator 130 to maintain a desired volage attenuation ratio even if the impedance of the ECG electrode that generates the input biopotential signal 129 changes. Impedance of an ECG electrode can change significantly (in some cases by an order of magnitude) during the period in which the patient is being monitored. Changes in electrode impedance can occur due to a number of factors, including perspiration by the patient, movement of the patient, electrodes being pulled away from the skin, or simply a loss of adhesion to the skin over time.

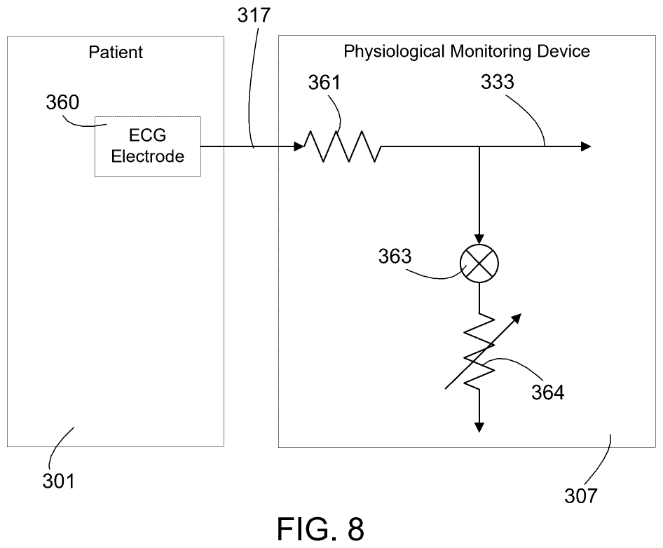

FIG. 8 shows an exemplary circuit that enables such adjustment. In this embodiment, the input biopotential signal moves through the ECG lead wire 317 from one of the ECG electrodes 360 (only one is shown in FIG. 8), then through a protection resistor 361. The signal then is passed to an attenuator resistor 364 having a switched electrical connection (via attenuator switch 363) with the protection resistor 361. The attenuated signal is then passed via lead 333 to the next circuit in the physiological monitoring device 307, such as a pass band filter or A/D convertor (see FIG. 2 and accompanying description above). When ESU filtration is inactive, the attenuator switch 363 is opened, which disconnects the attenuator resistor 364 from the circuit. In this embodiment, the attenuator resistor 364 is a variable resistor. As will be described below, this enables the physiological monitoring device 307 to maintain a desired voltage attenuation ratio for the input biopotential signal by compensating for change in impedance of the each of the ECG electrodes 360. In other examples, alternative methods could be used to compensate for changes in impedance of ECG electrodes 360, such as the use of an amplifier to buffer the signal.

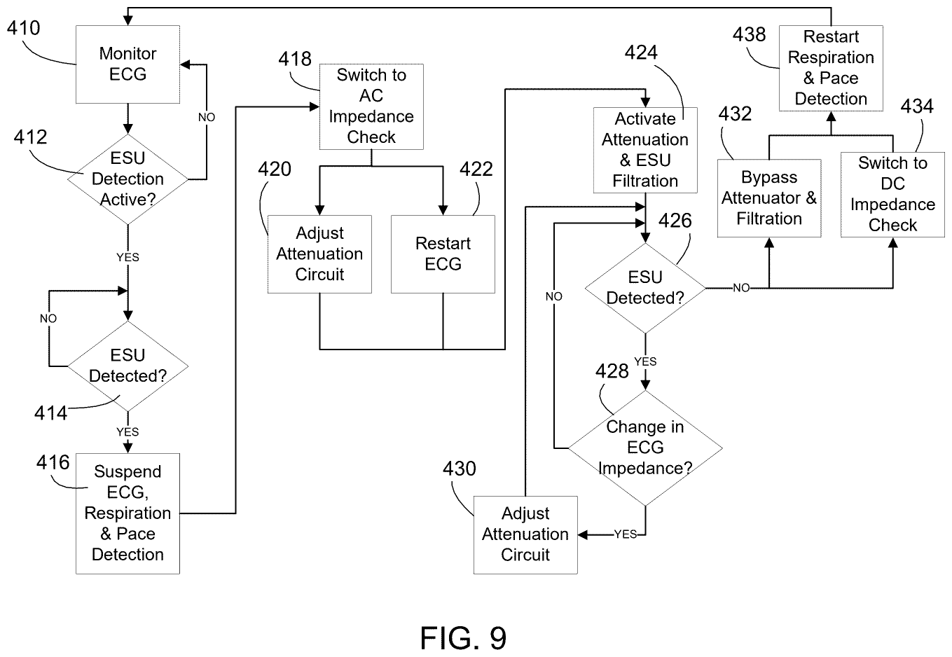

FIG. 9 is a flow chart showing an exemplary method of monitoring an ECG signal. It should be understood that this method could be applied to other types of biopotential signals. Impedance of each ECG electrode is monitored throughout the process. When ESU detection is inactive, DC impedance monitoring is used. As will be described herein, AC impedance monitoring is preferably used when ESU filtering is active, due to the higher accuracy of AC impedance monitoring. The ECG signal is monitored (step 410) until ESU detection is activated (step 412). As explained above, ESU detection could be activated in a number of ways, including manual user activation (e.g., via an icon on display/GUI) and automatic activation when the patient is determined to be located in an operating room. If ESU detection is active, then the system monitors input biopotential signals to determine if an ESU signal is present. When an ESU signal is detected, ECG monitoring, impedance respiration, and PACE detection are suspended (step 416). ECG monitoring is temporarily suspended until a more accurate impedance is measured. Impedance respiration and PACE detection interfere with AC impedance monitoring. Accordingly, these parameters are suspended until ECG electrode impedance monitoring is switched back to DC mode. ECG lead impedance measurement is switched to AC mode (step 418) and the impedance of each ECG electrode is measured. Upon conclusion of the impedance measuring step 418, the attenuation circuit is adjusted (step 420) to provide the desired voltage attenuation ratio based on the measured impedance and ECG monitoring is resumed (step 422). If the circuit of FIG. 8 is used, attenuation circuit adjustment is accomplished by adjusting the resistance of the attenuator resistor 364.

Attenuation of the input biopotential signal and filtration of the ESU signal is then activated (step 424). If the circuit of FIG. 8 is used, attenuation is activated by closing the attenuator switch 363. The system then periodically checks to determine if an ESU signal is still being detected (step 426).

If an ESU signal is still being detected, then it is determined whether ECG impedance has changed (step 428). In this example, remeasurement is triggered by a predetermined time interval (e.g., ECG impedance is remeasured every 5 minutes that step 424 is active). In other examples, rechecking of ECG impedance could be triggered by an event that is likely to cause a change in electrode impedance, such as movement of the patient, or an event that is likely to indicate a change in impedance, such as a significant change in the filtered ECG signal. If a change in ECG impedance is detected, the attenuation circuit is adjusted (step 430) to compensate for the change.

If an ESU signal is no longer detected, then the attenuator circuit and ESU filtration circuits are bypassed (step 432) and ECG electrode impedance measurement is switched to DC mode. Impedance respiration and PACE detection are then restarted (step 438) and the process is returned to step 410.

While various embodiments have been disclosed, it will be apparent to those skilled in the art that various changes and modifications can be made which will achieve some of the advantages of the concepts disclosed herein without departing from the spirit and scope of the present disclosure. It will be obvious to those reasonably skilled in the art that other components performing the same functions may be suitably substituted. It is to be understood that other embodiments may be utilized and structural or logical changes may be made without departing from the scope of the present disclosure. It should be mentioned that features explained with reference to a specific figure may be combined with features of other figures, even in those not explicitly mentioned. Such modifications to the general inventive concept are intended to be covered by the appended claims and their legal equivalents.

Furthermore, the following claims are hereby incorporated into the detailed description, where each claim may stand on its own as a separate example embodiment. While each claim may stand on its own as a separate example embodiment, it is to be noted that—although a dependent claim may refer in the claims to a specific combination with one or more other claims—other example embodiments may also include a combination of the dependent claim with the subject matter of each other dependent or independent claim. Such combinations are proposed herein unless it is stated that a specific combination is not intended. Furthermore, it is intended to include also features of a claim to any other independent claim even if this claim is not directly made dependent to the independent claim.

It is further to be noted that methods disclosed in the specification or in the claims may be implemented by a device having means for performing each of the respective acts of these methods. For example, the techniques described in this disclosure may be implemented, at least in part, in hardware, software, firmware, or any combination thereof. For example, various aspects of the described techniques may be implemented within one or more processors, including one or more microprocessors, DSPs, ASICs, or any other equivalent integrated or discrete logic circuitry, as well as any combinations of such components.

Further, it is to be understood that the disclosure of multiple acts or functions disclosed in the specification or in the claims may not be construed as to be within the specific order. Therefore, the disclosure of multiple acts or functions will not limit these to a particular order unless such acts or functions are not interchangeable for technical reasons. Furthermore, in some embodiments a single act may include or may be broken into multiple sub acts. Such sub acts may be included and part of the disclosure of this single act unless explicitly excluded.

1. A method of operating a patient monitor, the method comprising:
(a) receiving an input biopotential signal from at least one biopotential electrode that is electrically coupled to a patient, the input bipotential signal having an input voltage;
(b) attenuating the input biopotential signal to produce an attenuated biopotential signal having an attenuated voltage, the attenuated voltage being less than the input voltage;
(c) filtering the attenuated biopotential signal to reduce any interference generated by an electrosurgery unit (ESU), thereby producing a filtered biopotential signal;
(d) amplifying the filtered biopotential signal to obtain an output biopotential signal having an amplified voltage; and
(e) displaying the output biopotential signal.

2. The method of claim 1, wherein step (b) comprises attenuating the input biopotential signal by a predetermined voltage attenuation ratio.

3. The method of claim 1, wherein the input biopotential signal is an electrocardiogram (ECG).

4. The method of claim 1, further comprising:
(f) detecting the presence of an ESU signal.

5. The method of claim 1, further comprising performing step (b) before feeding the attenuated biopotential signal into a front end protective circuit of the physiological monitoring device, the front end protective circuit having a clipping voltage.

6. The method of claim 1, further comprising:
(f) processing the output biopotential signal before performing step (e).

7. The method of claim 1, wherein the input biopotential signal further comprises noise generated by the use of the ESU on the patient.

8. The method of claim 1, further comprising:
(f) measuring an impedance of each of the at least one biopotential electrode;
(g) adjusting an attenuator circuit based on the impedance measured in step (h) to maintain the predetermined voltage attenuation ratio.

9. A system comprising:
a sensor interface adapted to receive an input biopotential signal from at least one biopotential electrode that is electrically coupled to a patient, the input biopotential signal having an input voltage;
an attenuator electrically coupled to an output of the sensor interface, the attenuator being adapted to receive the input biopotential signal and to produce an attenuated biopotential signal having an attenuated voltage that is reduced from the input voltage by a voltage attenuation ratio;
an analog to digital convertor electrically coupled to an output of the attenuator, the analog to digital converter being adapted to convert the attenuated biopotential signal to a digital attenuated biopotential signal;
a protection circuit electrically coupled to an output of the attenuator, the protection circuit adapted to remove any portions of the digital attenuated biopotential signal that exceed a clipping voltage and to produce a clipped biopotential signal;
at least one filter electrically coupled an output of the protection circuit, the at least one filter adapted to reduce any ESU signal noise in the clipped biopotential signal and to produce a filtered biopotential signal, the ESU signal noise being generated by use of an electrosurgical instrument (ESU) on the patient when the input biopotential signal is being received;
an amplifier electrically coupled to an output of the at least one filter, the amplifier being adapted to receive the filtered biopotential signal and to produce an amplified biopotential signal having an amplified voltage that is increased from the attenuated voltage by a voltage amplification ratio; and
a display electrically coupled to the amplifier, the display being adapted to provide a visual representation of the amplified biopotential signal or a biopotential signal that is derived from the amplified biopotential signal.

10. The system of claim 9, wherein the attenuator is adapted to attenuate the input biopotential signal by a voltage attenuation ratio of at least 20.

11. The system of claim 9, wherein the sensor interface comprises an electrocardiogram (ECG) sensor interface.

12. The system of claim 9, further comprising an ESU detection unit having an active state and an inactive state, the ESU detection unit being adapted to detect the presence of an ESU signal in the input biopotential signal only in the active state.

13. The system of claim 9, wherein the attenuator includes a variable resistor.

14. The system of claim 9, further comprising an AC impedance detection circuit adapted to measure the impedance of each of the at least one biopotential electrode.

15. A method comprising:
(k) receiving an input biopotential signal from at least one biopotential electrode that is electrically coupled to a patient and a physiological monitoring device, the input bipotential signal having an input voltage;
(l) detecting the presence of an interference signal in the input biopotential signal;
(m) attenuating the input biopotential signal by a predetermined voltage attenuation ratio to produce an attenuated biopotential signal having an attenuated voltage, the attenuated voltage being less than the input voltage and less than a clipping threshold of a protective circuit of the physiological monitoring device;
(n) filtering the attenuated biopotential signal to remove at least one component of the interference signal, thereby producing a filtered biopotential signal;
(o) amplifying the filtered biopotential signal to obtain an output biopotential signal having an amplified voltage; and
(p) displaying the output biopotential signal.

16. The method of claim 15, further comprising:
(q) performing steps (c) through (e) only when an interference signal is detected in step (b).

17. The method of claim 15, further comprising:
(r) measuring an electrode impedance for each of the at least one biopotential electrodes; and
(s) adjusting an attenuator resistance based on the measured electrode impedance in order to achieve the predetermined voltage attenuation ratio.

18. The method of claim 15, step (c) further comprises attenuating the input biopotential signal by a predetermined voltage attenuation ratio to produce an attenuated biopotential signal having an attenuated voltage, the attenuated voltage being less than the input voltage and less than a clipping threshold of a protective circuit of the physiological monitoring device.

19. The method of claim 15, wherein the at least one biopotential electrode is an ECG electrode.

20. The method of claim 15, wherein step (c) comprises attenuating the input biopotential signal by a predetermined voltage attenuation ratio of at least 20.
